(19) United States
(12) Patent Application Publication
(10) Pub. No.: US 2008/0317810 A1
(43) Pub. Date: Dec. 25, 2008

(54) Methods and Compositions for Reducing Oxalate Concentrations

(76) Inventors: Harmeet Sidhu, Gainesville, FL (US); Clarence C. Lee, Atlanta, GA (US)

Correspondence Address: TROUTMAN SANDERS LLP, 600 PEACHTREE STREET , NE, ATLANTA, GA, 30308, US

(21) Appl. No.: 11/659,583

(22) PCT Filed: May 9, 2005
(86) PCT No.: PCT/US05/16080

§ 371 Date: Mar. 19, 2008

Related U.S. Application Data
(60) Provisional application No. 60/569,337, filed on May 7, 2004.

(51) Int. Cl. A61K 9/00; A61K 38/44; A61P 13/12

(52) U.S. Cl. 424/423; 424/94.4

(57) ABSTRACT

The present invention provides methods and compositions for oxalate degradation. The present invention comprises devices comprising oxalate-reducing enzymes. The enzymes may be directly attached or incorporated in coatings on the devices. Such devices are useful in treatments where oxalate or oxalic acid deposits interfere. Methods for making and using such medical devices are also included.

### RELATED APPLICATIONS

This application claims the priority of U.S. Provisional Patent Application No. 60/569,337, filed May 7, 2004, and is a continuation-in-part of U.S. patent application Ser. No. 10/266,718, filed Oct. 7, 2002, which claims the priority of U.S. Provisional Patent Application No. 60/327,544, filed Oct. 5, 2001, all of which are incorporated herein by reference.

### TECHNICAL FIELD

The present invention relates to methods and compositions for reducing oxalate concentrations. In particular, the invention relates to methods and compositions used for devices that provide oxalate reducing activity to environments.

### BACKGROUND

Oxalic acid, or its salt, oxalate, is a natural by-product of metabolic processes in vertebrate animals and many consumable plants. Unfortunately, oxalate is not properly degraded in a significant portion of humans, a condition which may result in the formation of kidney stones in those persons. It is estimated that 70% of all kidney stones are composed of oxalate. Approximately 12 percent of the U.S. population will suffer from a kidney stone at some time in their lives. Persons suffering from, and at risk for, developing kidney stones, as well as patients with lipid malabsorption problems (e.g., sprue, pancreatic insufficiency, inflammatory intestinal disease, bowel resection, etc.), tend to have elevated levels of urinary oxalate.

During end-stage renal disease (ESRD), oxalate accumulates resulting in hyperoxalemia and secondary oxalosis (1). To provide the functions of a normally functioning kidney, hemodialysis provides clearance of plasma oxalate and plasma calcium oxalate. However, hemodialysis treatments do not totally eliminate the oxalate and calcium oxalate (2). Without hemodialysis, patients with ERSD and primary hyperoxeluria (PH) have a higher risk of progressive systemic oxalosis (3).

In addition to the formation of stones in the urinary tract, the presence of oxalate in fluids can be problematic in a number of situations. For example, it is well established that the formation of calcium oxalate deposits on stents and catheters can compromise the ability of these devices to perform their functions, and can be a contributing factor in the establishment of microbial infections. Similarly, the formation of oxalate deposits also contributes to reduced efficiency and spoilage in fermentation processes, including in the brewery industry.

There are deleterious affects of oxalate on indwelling catheters and stents. Catheters and stents are commonly used in medical procedures including, for example, for the management of urinary and peritoneal dialysis flow, haemodialysis, and drainage of renal calculi after laser or lithotripsy treatment. The use of long-term indwelling urinary catheters and stents in the urinary tract suffers limitations due to the occurrence of encrustation and bacterial adhesion which increases the risk of blockage of the device and urinary tract infections (UTI).

The pathogenesis of UTI associated with urinary devices often involves the formation of biofilms. An important step in the biofilm formation is the deposition of urinary components onto the device in the form of a conditioning film. (4-6). Within hours, this film can consist of encrustation deposits that interfere with the urine flow. Biochemical and optical analyses of such encrustation has revealed the presence of calcium oxalate.

Epidemiological studies on the incidence of stent encrustation in various urological conditions have indicated urolithiasis as a major risk factor for stent encrustation. A significantly higher incidence of encrustation has been reported in stone formers (7,8). Keane et al. report encrustations on 58% of stents and the major risk factor for stent encrustation was a history of urolithiasis. This is apparently due to supersaturation of a stone former's urine with the lithogenic components mainly calcium and oxalate. The oxalate concentration of urine is the most important parameter for calcium oxalate saturation. Since there are >10 calcium ions for each oxalate ion in urine, small increases in oxalate result in exceeding the threshold of calcium oxalate supersaturation causing its crystallization.

As patients usually receive antibiotics prior to placement of indwelling medical devices the normal urethral flora has often been replaced with uropathogenic drug resistant organisms adept at colonizing surfaces. The presence of a catheter or a stent can also facilitate the ascent of the bacteria into the renal pelvis and tissues. Once device-related infections become established they are generally difficult to treat and usually necessitate the removal of the device.

In addition to being a site of infection, a large number of stents have been shown to have impassable blockage (9). Encrustation deposition on synthetic materials in the urinary tract can occur both in infected and sterile urine. The mechanism of encrustation in infected urine is analogous to formation of urinary stones: urease producing organisms elevate the urinary pH by hydrolysis of urea thereby creating an alkaline environment where magnesium and calcium readily precipitate out of solution forming crystals.

In sterile urine, the formation of encrustation on the biomaterials appears to be dependent on both urinary constituents and the properties of the synthetic material (10,11). A wide variety of ureteral stents and urethral catheters are available. Silicone is a highly biocompatible material and is used for the manufacture of urethral catheters as well as ureteral stents. Although there has been research into the development of materials with modified surface properties, none of these has been established to be encrustation resistant (12). Some of the commonly used devices are the standard silicone latex device (Sof Flex, Cook Urology, Indiana), hydrophilic stents (Boston Scientific, MA) and the low surface energy stents (LSe, Cook Urology, IN) that have been specially designed to lower the risk of encrustation. Extensive studies carried out in the Urology ESWL Center at University of Western Ontario have documented encrustation and biofilm processes on all the above commonly used biomaterials (5, 13-15).

One of the more recent developments is the use of hydrogel coatings on indwelling medical devices. These hydrophilic polyurethane polymers swell on contact with water and retain a significant proportion of water within their polyanionic structure. This is believed to reduce friction and stent encrustation. Studies have also been undertaken with non-ionic synthesized hydrogels, such as polyacrylamide, polyvinyl alcohol, polyethylene glycol and polymethoxy-PEG methacrylate and with ionic hydrogels such as crosslinked polyacrylamide-dimethyl-aminoethyl methacrylate copolymers (16). A polyethylene glycol-based hydrogel linked to serum albumin has been reported to be a suitable matrix for enzyme immobilization on the biomedical devices (17).

Surface immobilization of enzymes is a convenient and cost-effective method for allowing simple recovery and reuse of an enzyme in industrial and laboratory settings. Enzymes have been covalently linked to substrates using a variety of bioconjugation techniques. Common substrates include organic materials such as polycarbonate and polysaccharide materials (including chitosan) and inorganic materials such as silica glass. These substrates are often surface functionalized in order to covalently link enzymes.

Silicone elastomer is a commonly used material in urinary catheter manufacturing because of its bioinertness, low coefficient of friction, and flexibility. Attempts have been made to modify the silicone elastomer surface in order to produce a more hydrophilic (18-20), functionalized (21-23), or graft polymerized (20, 24-27) surface. Surface modification has been conducted in various ways including chemical reaction, UV irradiation, gamma irradiation, and radio-frequency plasma discharge (RFPD). RF plasma discharge is a useful technique for changing the surface properties of a material without modifying the bulk properties in order to enhance biocompatibility, such as changes in protein adsorption, or it can be used to increase desirable cell adhesion and growth to the biomaterial surfaces (20, 26-28).

Many of the recent advances in the development of indwelling medical devices are focused on the prevention of bacterial infection. Coating or impregnating the biomaterials with antimicrobial substances such as heavy metals, silver oxide and antibiotics could provide reduction in the incidence of infection especially for patients requiring short term insertion of a device (29, 30).

In the fermentation industry, scale is formed by the surface deposition on equipment of 1) water-insoluble sulfate and carbonate salts of calcium and magnesium (mineral scale) and 2) calcium oxalate that forms during the normal fermentation process. The presence of scale on equipment is not serious if addressed early. If left unchecked, however, it can result in significant operational problems.

Scale represents a microbiological hazard. The crystalline structure of scale affords significant protection for spoilage organisms (such as Pediococcus and Lactobacillus) from the effects of cleaning and sterilizing agents. Second, scale formation significantly reduces heat transfer across equipment surfaces. This in turn reduces the efficiency of heating and cooling operations, which translates into increased turn-around time, reduced production capacity, and inflated energy costs. Third, scale is especially detrimental to boilers, because it is rapidly deposited under the harsh conditions in which a boiler operates. This buildup can lead to boiler failure and possible explosion.

Unfortunately, the chemical reactions that lead to scale formation are inherent to the fermentation process. For example, scale is formed via a number of chemical reactions that occur throughout the brewing process. One form of scale, commonly known as beer stone, is brought about by the reaction of calcium with oxalic acid under the conditions of mashing. Oxalic acid is an organic acid constituent of barley. In addition to scale formation, calcium oxalate has been linked to gushing and colloidal instability of packaged beer. There are a number of options for dealing with scale in the brewery. These include ion exchange, sequestering agents, and acid cleaners.

What is needed are medical devices and other materials that are capable of resisting the formation of biofilms, encrustation and incrustion. In the field of urinary medical devices, what is needed are medical devices that are capable of lessening or preventing oxalate encrustation or incrustation.

### SUMMARY OF THE INVENTION

The invention comprises materials and methods for reducing oxalate concentrations in fluids. In an aspect of the invention, oxalate-reducing enzymes are immobilized on a surface and provide an efficient means of lowering free oxalate in a fluid which comes in contact with the surface. In specific embodiments exemplified herein, one or more types of oxalate-reducing enzymes are attached to surfaces or entrapped within polymeric matrices which are contacted with biological fluids. These surfaces or matrices may be on, for example, catheters, stents, or dialysis membranes.

In a further embodiment described herein, oxalate-reducing enzymes are attached to surfaces which come into contact with fluids involved in a fermentation process. These modified surfaces reduce the incidence of scale formation during the fermentation processes.

As described herein, oxalate-reducing enzymes are coated onto materials used for the manufacture of devices for an efficient in situ degradation of oxalate to prevent the initial steps of calcium oxalate precipitation leading to encrustation. This approach can be used to prevent the blockage of the devices and the risk of microbial infection.

In one embodiment of the subject invention, radio-frequency plasma discharge (RFPD) is used to activate and functionalize an inert silicone elastomer surface. The surface is then coated with 3-aminopropyltriethoxysilane (AMEO) to derivatize the elastomer surface with an amine functionality to provide a site for coupling oxalate oxidase amine groups. In another embodiment, oxalate-reducing enzymes are incorporated in a hydrogel that is then coated on a surface, such as a stent or other medical device, that is resistant to incrustation and encrustation.

### DETAILED DESCRIPTION

The present invention comprises methods and compositions for oxalate reduction. In particular, the present invention comprises methods for making medical devices comprising oxalate reducing capability. The oxalate reducing capability can be provided on the medical device by the attachment of on or more types of oxalate-reducing enzymes to the medical device. Such medical devices include, but are not limited to, catheters, stents, artificial bladders, sutures, and filtering devices. The attachment of the oxalate-reducing enzymes can be directly to the medical device or to a coating on the medical device. The oxalate-reducing enzyme can be attached via the methods described herein.

Aspects of the present invention comprise oxalate reducing enzymes entrapped by a polymeric matrix. The enzymes are physically entrapped by the polymeric material. The polymeric material may immobilize the enzyme or the polymeric material may control the release of the enzyme into the environment surrounding the polymeric matrix. The polymeric matrix comprising the oxalate reducing enzymes may be made into a medical device, may make up a portion of a medical device or may form a coating on a medical device. The polymeric matrix may be found in any shape necessary for the functioning of the enzymes in the environment.

The term oxalate-reducing enzyme includes all types of oxalate-reducing enzymes, and is not limited to, oxalate oxidase, oxalate decarboxylase, and oxalyl-CoA decarboxylase, and includes enzymes that are capable of interacting with oxalate or oxalic acid. The term also includes enzymes referred to as oxalate-degrading enzymes. The enzymes may be isolated and used in the present invention as individual enzyme proteins or may be associated with bacteria which express such enzymes, either naturally or through recombinant methods. As used herein, the term oxalate-reducing enzymes includes enzyme proteins or bacteria comprising such oxalate-reducing enzymes. The present invention contemplates the use of one or more types of enzymes in the compositions and devices taught herein. These enzymes may be derived from natural sources or synthesized using recombinant means, and include all fragments, such as binding sites, active sites, or fragments capable of interacting with oxalate or oxalic acid. This term also includes all necessary cofactors, coenzymes, metals, or binding or substrate materials that are needed by the enzyme in interacting with oxalate or oxalic acid. The present invention also contemplates any binding partners of these enzymes and includes antibodies and antibody fragments that bind to or interact with the enzymes.

The subject invention provides materials and methods for reducing the oxalate concentration in fluids. This reduction in oxalate concentration is achieved by reducing the oxalate, or otherwise removing oxalate from the fluid. Methods of the present invention comprise providing materials comprising oxalate reducing enzymes wherein oxalate is degraded in biological fluids or other fluids in which oxalate is found. Specifically exemplified herein are stents and catheters which have been modified to reduce oxalate concentrations in surrounding fluids thereby reducing or eliminating encrustation.

A further aspect of the subject invention comprises materials incorporating or associated with oxalate reducing enzymes, such as dialysis membranes which have been modified to degrade oxalate thereby reducing the oxalate concentration of the surrounding fluid and increasing the efficiency of the dialysis procedure.

Methods also comprise providing materials wherein oxalate is degraded in fluids including fermentation broths. For example, oxalate is found in fluids involved in the brewery process, and materials of the present invention can be added during the brewing process to reduce or degrade the oxalate found in fermentation broths.

The present invention comprises modifying surfaces which come into contact with a fluid and such surfaces are modified by associating, attaching, coating, adhering oxalate-reducing enzymes with these surfaces. The surfaces which are modified in accordance with the subject invention may be, for example, made of a polymeric material. In one embodiment, the surface comprises silicone rubber.

The methods of the subject invention are distinct from the attachment of enzymes to surfaces for the purpose of conducting assays. Thus, the methods of the subject invention provide advantages through the reduction of the oxalate concentration in a fluid. Methods of the present invention comprise prevention or treatment of surfaces or liquids to provide a reduction in the formation of calcium oxalate deposits, microbial infections, encrustation, bacterial adhesion, and scale formation.

In an aspect of the present invention, the association of the enzymes with the surface may be by, for example, direct attachment, attachment through a linker, or by incorporation of the enzymes within a coating on the surface. The present invention contemplates use of methods known in the art for attachment of proteins to surfaces for providing oxalate-reducing enzymes on a particular surface. The present invention contemplates that the activity of the attached enzymes is not significantly impaired and that the enzymes function to degrade oxalate. For example, methods are known to those skilled in the art for directly attaching enzymes to synthetic materials. Alternatively, the enzymes may be attached to the desired surface through a linker. A number of such linkers are known to those skilled in the art and include dendrimers such as those described in U.S. Pat. No. 6,080,404.

In an aspect of the present invention, oxalate-reducing enzymes are entrapped within a coating. The coating may be, for example, those described in U.S. Pat. Nos. 5,554,147; 5,607,417; and 5,788,687. These patents, which are incorporated herein by reference in their entireties, also describe various agents which can be entrapped within a device coating and used in conjunction with the oxalate-reducing enzymes of the subject invention.

An aspect of the invention comprises polymeric materials, also referred to herein as polymeric matrices, that physically immobilize oxalate reducing enzymes or such polymeric matrices may control the rate of release of oxalate reducing enzymes into the environment around the matrix. Such matrices comprise natural polymers, synthetic polymers, biopolymers or mixtures or combinations of these polymers. Biopolymers include the polymeric molecules that are synthesized by living organisms, and includes such polymers made by synthetic or chemical methods or isolated from the organisms. Examples of biopolymers include, but are not limited to, proteins, polysaccharides, mucopolysaccharides, heparin, heparin sulfate, heparinoids, dermatan sulfate, pentosan polysulfate, chondroitin sulfate, cellulose, agarose, chitin, carrageenin, linoleic acid, and allantoin, cross-linked collagen, fibronectin, laminin, elastin, cross-linked elastin, collagen, gelatin, hyaluronic acid, chitosan alginate, dextran, methylcellulose, DNA, RNA, other nucleic acid polymers, polylysine, and natural rubber. Such polymeric matrices are porous such that small water soluble molecules can enter and exit the polymeric coating, including, but not limited to molecules such as oxalate, formic acid, formate, carbon dioxide, oxygen, or oxalyl-CoA.

An aspect of the invention comprises entrapping oxalate reducing enzymes in a polymeric matrix that forms a coating on an article. Such as article may be any form, may be a medical device, including but not limited to stents, catheters, bladders, invasive devices in contact with body fluids, or may be microparticles or nanoparticles. Such articles may be dipped or coated using methods known in the art with a polymeric matrix comprising active oxalate reducing enzymes.

Although research is being done on biomaterial modifications of stents and catheters, none of the currently used devices reduces the levels of encrustation satisfactorily. The methods and devices of the subject invention can be used in patient populations undergoing endourological procedures. These methods and devices can be used by patients who are prone to oxalate stone formation and those suffering from advanced malignant obstruction.

Silicone rubber (SR), is used for many biomedical devices because it has excellent biocompatibility, provides flexibility and long-term mechanical stability within the physiological environments. To prevent rapid deposition of a conditioning film in the urinary environment, hydrogel coatings are applied to SR devices. Several techniques have been developed for the surface modification of SR via covalent grafting of various polymers (20, 27, 31, 32). The surface of SR can be activated to provide functional groups capable of linking to polymerizable monomers. Activation can be accomplished using conventional energy sources, such as cobalt-60, radiofrequency or microwave gas discharge, and plasma discharge. Additionally, photochemical reactions using UV and redox reagents have been used to chemically modify an SR surface.

With argon-plasma treatment, radicals are produced on the silicone surface, and then functional groups are produced by exposing the sample to gases, such as oxygen to produce peroxides, and ammonia to produce amide groups. Plasma treatment technique can be used to produce surface hydroperoxides to initiate graft polymerization of polyacrylic acid (PAA) and polyglycidylmethacrylate, and a method to covalently immobilize fibronectin to activated PAA is known (27). The present invention contemplates use of this and other methods for covalently immobilizing oxalate reducing enzymes directly to the SR and to a hydrogel matrix of PAA.

SR can also be modified with a polyethylene glycol (PEG)-gelatin hydrogel. In one embodiment, this coating can be used to entrap an active agent such as antibiotics, antimicrobial agents, antifungal agents, antibacterial agents, anti-viral agents, antiparasitic agents, anesthetics, growth factors, angiogenic factors, tissue healing agents, adjuvants, antibodies, or antibody fragments, and combination or mixtures of one or more of these. Examples of antibiotics which can be used include fluoroquinolones, β-lactams, and cephalosporins. Examples of active agents include, but are not limited to antimicrobial agents such as isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, bromide, iodide and periodate, growth factors, include but are not limited to basic fibroblast growth factor, acidic fibroblast growth factor, nerve growth factor, epidermal growth factor, insulin-like growth factors 1 and 2, platelet derived growth factor, tumor angiogenesis factor, vascular endothelial growth factor, corticotropin releasing factor, transforming growth factors α and β, interleukin-8, granulocyte-macrophage colony stimulating factor, interleukins, and interferons. The active agent may be entrapped within liposomes or other delivery vehicles. A combination of one or more active agents along with the oxalate-reducing enzymes entrapped within a coating can address catheter encrustation, of bacterial-induced deposits of struvite and calcium phosphate, and calcium oxalate deposits from a non-infected environment.

In one embodiment of the subject invention, oxalate-reducing enzymes can be covalently bound to a radio-frequency plasma surface-modified silicone elastomer. These surface-modified materials can be used, for example, in the prevention of calcium oxalate encrustation on urological biomaterials. Though not wishing to be bound by any particular theory, it is believed that the oxalate-reducing enzyme affects the microenvironment surrounding the surface of the catheter and prevents the formation of calcium oxalate crystals at the surface. In addition, the production of H₂O₂, through OXO-mediated (Oxalate oxidase) free oxalate degradation, may provide an antimicrobial affect in the urinary environment.

An aspect of the present invention comprises urinary devices comprising oxalate-reducing enzymes. Such devices can be used in treatments of ureter injuries and kidney stone formation and in treatment or prevention regimens requiring the implantation of such devices. One aspect of the invention is to provide devices comprising oxalate-reducing enzymes immobilized on or within the surface of the device, wherein the device and/or the enzymes retain biological activity for at least four weeks, for at least twelve weeks, and/or for at least twenty six to fifty two weeks. An aspect of the invention is to provide devices comprising oxalate-reducing enzymes immobilized within a polymeric matrix, or the oxalate reducing enzymes are released in a controlled fashion from the polymeric matrix, wherein the polymeric matrix is the surface of the device, wherein the enzymes retain biological activity for at least four weeks, for at least twelve weeks, and/or for at least twenty six to fifty two weeks. Devices of the present invention, such implanted materials comprising oxalate-reducing enzymes retain biological activity for at least four weeks, for at least twelve weeks, and/or for at least twenty six to fifty two weeks.

A number of different materials are used in the manufacture of urinary devices. The most common material, used for example in Foley catheters, is latex rubber. Its properties, namely the high surface free energy, high elasticity, low coefficient of friction, and low production costs, make it a very suitable material for device production. Latex rubber has drawbacks, primarily the presence of cytotoxic-contaminating substances from the source material, and patient sensitivity to allergens present in latex.

Another common material used in urinary device manufacture is silicone rubber made up of a class of polymers called siloxanes. Siloxanes are silicon-based polymers which contain a —(Si—O)— backbone with organic (methyl, phenyl, etc.) groups bonded to the silicon atoms. The main silicone form used to manufacture biomedical implants is polydimethylsiloxane (PDMS), which exists in three forms—oils, gels, and elastomers—depending on the level of crosslinking agent and filler added in processing. PDMS is a hydrophobic polymer, and is commonly reinforced with fused silica (SiO₂) to produce a strong, elastic material. Silicone rubber is extremely flexible, stable in the urinary environment, bioinert, and has a low coefficient of friction.

Polyurethanes are also used for urinary devices. Polyurethanes are copolymers composed of a backbone with urethane linkages [—OC(O)NH—], linking “hard” and “soft” segments which phase separate into a micro-domain morphology. This phase segregated morphology provides physical cross linking to the copolymer, which is reinforced by the secondary hydrogen bonding from the urethane linkage, providing good mechanical properties to the elastomer. However, polyurethanes, such as polyester polyurethanes and polyether polyurethanes, are more sensitive to the urinary environment than silicones due to hydrolytic degradation. Polyurethane is a common generic class of synthetic polymers with strength property between that of silicone and polyethylene. It is stiffer than silicone but more elastic than polyethylene. Although silicone and polyurethane are the most commonly used biomaterials in stent manufacturing, recently certain proprietary polymers, for example, C-flex (Concept Polymer Technologies, Clearwater, Fla.), Silitek (Medical Engineering Corporation, Racine, Wis.) and Percuflex (Boston Scientific Corporation, Watertown, Mass.) have been used.

The present invention comprises medical devices, such as catheters, implants and stents with hydrogel coatings that provide oxalate-reducing capability. While hydrogels exhibit a low coefficient of friction and are flexible, they generally lack the mechanical strength needed for an article such as a stent or catheter, and are used as surface coatings on substrates. The surface coating is often applied in a dip-coating process, in the presence of a crosslinking agent, and then the coating is allowed to cure onto the substrate. Commonly used hydrogel monomers include 2-hydroxyethyl methacrylate (HEMA), acrylamides, n-vinyl-2-pyrrolidone (NVP), and methacrylic acid (MAA). Hydrogel coatings are known for their ability to absorb several times their weight in water, becoming soft and lubricious. Adding a hydrogel surface to an implant device produces an article that exhibits bulk mechanical properties (flexibility) of the substrate material and improved biocompatibility of the surface coating.

Hydrogel coatings provide an environment to immobilize enzymes. The high water content of the swollen hydrogel provides a microenvironment for water-soluble enzymes. Hydrogels may show reduced conditioning film adsorption, thereby allowing the enzyme greater access to the substrate molecules. A PEG-based hydrogel, in which PEG is linked to serum albumin, has been reported to be a suitable drug-controlled delivery device and a matrix for enzyme immobilization on biomedical devices. The cross-linking of PEG with albumin provides some mechanical stability to the polymerized hydrogel. In devices of the present invention, hydrogel coatings, comprising one or more oxalate reducing enzymes, are applied to a silicone or polyurethane elastomer stent after appropriate surface modification.

The present invention comprises methods and devices comprising surface modifications, which include many alternative methods for altering the surfaces. The long-term usefulness of a urinary device is dependent on its response to interfacial interactions. The surface physico-chemical characteristics, such as composition and functionality (polar/apolar, H-bonding, ionic charges), topography (roughness, porosity, gas micro-bubbles), and hydrophobicity, are factors in the interactions between the device surface and the surrounding biological environment. The first few Ångstroms to nanometers of the surface characteristics form the interface between the device and the environment.

Attempts have been made to modify the surface of biomaterials, such as those used in medical devices, in order to improve biocompatibility, namely, to make the material inert to the surrounding body tissues, or to stimulate intercalation in cases where attachment is desired. Biomaterial surface modifications not only improve the biocompatibility but can also provide surface functionality to increase retention of biomolecules (proteins or antibiotics) on the device surface or provide a surface for the attachment of a delivery system. For example, the present invention comprises a medical device having a surface coated with a hydrogel comprising incorporated enzymes and/or active agents such as antibiotics. Surface modification can be achieved by chemical or physical techniques. Each of these techniques offers the advantage of tailoring the polymer surface properties, while the desirable bulk mechanical properties remain constant.

The present invention comprises devices comprising physically modified surfaces. Physical surface modification of a polymer generally alters the bulk material itself, or deposits a layer on top of the bulk material. Physical modification methods for biomaterials include, but are not limited to, cold gas plasma treatments, ultraviolet (UV) irradiation, laser treatments, gamma irradiation, and physical vapor deposition (PVD) techniques, which include such methods as thermal evaporation, sputtering, and laser ablation of a target material to produce a flux of coating materials that are then deposited on the substrate. To achieve the desired surface characteristics, such as being hydrophobic or hydrophilic, having an increased surface area, or an increase in functional groups for further modification, the thickness of the layer on the substrate can be varied by varying the deposition rate or the length of the PVD process.

The pulsed laser-assisted surface functionalization (PLASF™) technique, described herein, involves physical vapor deposition of a polymer coating onto a surface for improved chemical attachment of a secondary species, such as the oxalate-reducing enzymes. The PLASF™ process produces a highly uniform, ultra-thin coating that requires minimal processing steps. Both gas plasma treatment and PLASF™ were used for surface modification of medical grade silicone are described herein. The PLASF™ treatment was found to be more effective than gas plasma treatment in maintaining the stability and functionality of the immobilized enzyme in in vitro assays. Similar methods are used to immobilize the oxalate-reducing enzymes in a hydrogel coating deposited using PLASF™.

The present invention comprises devices comprising chemically modified surfaces. Chemical modification methods include surface oxidation, hydrolysis, functionalization, and surface grafting of macromolecules via covalent bonding. Surface grafting is useful for the attachment of hydrophilic hydrogel coating onto devices such as stents. Surface grafting can be achieved by coupling the hydrogel components or oxalate-reducing enzymes to chemically-reactive surface functional groups or by the introduction of functional groups via chemical treatments, including the irradiation techniques listed above. Radiation-induced graft polymerization can be used to effectively control the morphology, structure, and thickness of the grafted layer, and is commonly achieved using ultraviolet (UV) light, to initiate the polymerization via free-radical initiators. Graft polymerization can also be achieved by use of gamma irradiation, such as in Ciba Vision's Hydrograft™, in which the silicone elastomer surface is suspended in a neutral or ionic water soluble, hydrophilic, vinylic monomer, such as like: HEMA, NVP or MAA, containing the oxalate reducing enzyme. Gamma irradiation of this elastomeric surface results in formation of a graft polymerized coating with entrapped bioactive drug or enzyme. The present invention comprises using chemical surface modification methods to apply the enzyme-hydrogel coating to surfaces. Methods of chemical modification are especially useful in modifying both the internal and external lumen of tubular devices, and lower initial costs since it does not require specialized apparatus.

### Oxalate Reducing Enzymes

There are three main classes of oxalate reducing enzymes. Oxalate oxidase, is expressed in higher plants and it catalyzes the oxygen dependent oxidation of oxalate to CO₂ with concomitant formation of H₂O₂ A rapid three step purification procedure has been developed to obtain oxalate oxidase from barley roots (33). The gene encoding the barley root oxalate oxidase has been cloned, sequenced and expressed (34).

Oxalate decarboxylase, the second class of oxalate metabolizing enzymes, is mainly present in fungi (35). Fungal oxalate decarboxylase catalyzes the degradation of free oxalate to CO₂ and formate. This enzyme has been reported in several fungi, including Myrothecium verrucaria, certain strains of Aspergillus niger, and white rot fungus, Coriolus versicolor. The gene encoding the Flammulina velutipes oxalate decorboxylase has been cloned and sequenced (36); International patent no. WO 98/42827.

The bacterial enzyme for oxalate degradation, oxalyl-CoA decarboxylase, is active on the CoA-activated substrate and converts it into formyl-CoA. A formyl-CoA transferase then acts to exchange formate and oxalate on CoA. These enzymes have been studied in the oxalate reducing bacteria, Pseudomonas oxalaticus present in the soil (37) and in Oxalobacter formigenes, residing in the gastro-intestinal tract of vertebrates, including humans (38). O. formigenes has been shown to play a symbiotic relationship with its hosts by regulating oxalic acid absorption in the intestine as well as oxalic acid levels in plasma. As a result the absence of this bacteria has been found to be a risk factor in oxalate related disorders like recurrent idiopathic calcium oxalate urolithiasis (39, 40) and enteric hyperoxaluria secondary to jejuno-ileal bypass surgery, cystic fibrosis and inflammatory bowel disease (41, 42). This bacterium is highly specific in utilizing only oxalate as its source of energy for its survival and growth. As a result the oxalate reducing enzymes oxalyl-CoA decarboxylase and formyl-CoA transferase comprise about 20 to 30% of its cellular proteins. Both the proteins as well as the membrane transporter for the oxalate-formate exchange have been purified and well characterized (43-45). Also, the genes for all three proteins have been cloned, sequenced and expressed as biologically active recombinant proteins (46-48). Patents describing various oxalate-reducing enzymes and the genes encoding these enzymes include U.S. Pat. Nos. 5,912,125; 6,090,628; and 6,214,980. These patents are incorporated herein by reference.

The present invention comprises devices comprising one or more oxalate-reducing enzymes. These enzymes can be derived from sources known to those skilled in the art. For example, the plant enzyme, oxalate oxidase (OXO) can be obtained commercially (Sigma Chemical Co., St. Louis, Mo.), as a lyophilized powder, and reconstituted in 50 mM sodium succinate buffer, pH 4.0 for use with coated devices.

Alternatively the oxalate-reducing enzymes can be derived by recombinant means. For example, recombinant means such as cloning, expression and purification were used to obtain the B. subtilis oxalate decarboxylase enzyme. The present invention contemplates using one or more oxalate-reducing enzymes in association with devices, and the selection of the one or more enzymes depends on such characteristics as enzymatic activity and stability after immobilization on the device, in the environment where the device is used

Such recombinant methods are known to those skilled in the art. For example, disclosed, in general, is the cloning and expression of B. subtilis oxalate decarboxylase (YvrK) gene. The gene for oxalate decarboxylase protein (YvrK) has been cloned into the pET-14b plasmid (Novagen, Wis.), under the control of a strong bacteriophage T7 promoter, for over-expression as soluble cytosolic protein. The expression host was the E. coli strain BL 21(DE3) pLysS, a λDE3 lysogen deficient in proteases and which contains a chromosomal copy of the T7-RNA polymerase gene under the lacUV5 control. In addition, this strain carries a pET-compatible plasmid that encodes T7 lysozyme, a bifunctional enzyme that cuts a bond in the peptidoglycan layer of the cell wall and inhibits T7 RNA polymerase. This enables greater control of uninduced basal expression and allows the use of methods that disrupt the inner membrane (freeze-thaw, mild detergents, etc.) to efficiently lyse the cell. Expression of the gene product is induced by the addition of isopropyl-β-D-thiogalactopyranoside (IPTG).

Purification of oxalate decarboxylase expressed as a recombinant protein in E. coli was performed using His-Bind columns and buffers from Novagen. A small-scale (<10 mg per column) purification of oxalate decarboxylase involved metal affinity chromatography on pre-charged His-Bind columns followed by elution using an increasing imidazole concentration to competitively displace the His-tagged recombinant protein. Briefly, cells were harvested from an induced culture after 2-3 hours of IPTG induction. The cells were resuspended in Lysis Buffer (1× Binding Buffer+0.1% Triton X-100) and sonicated on ice. The soluble filtered fraction was loaded onto a column equilibrated with 1× Binding Buffer (5 mM imidazole). The column was then washed once with 1× Binding Buffer, once with 1× Wash Buffer (60 mM imidazole) and finally, the bound target protein was eluted with 1× Elution Buffer (1M imidazole). The expressed protein bound very well to the affinity column as shown by absence of the expressed protein in the flow through and column wash-out fractions. The majority of the expressed protein was then eluted off the column. The eluted fraction was concentrated using Centriprep ultracentrifugation columns.

Scaling up the purification of recombinant oxalate decarboxylase required a resin that would be compatible with the high flow rates. Ni-NTA His-Bind Superflow (Novagen) with a binding capacity of 5-10 mg protein/ml resin fits these requirements. The protein elution strategy utilizing increasing imidazole concentrations to competitively displace the His-tagged recombinant protein was optimized using this resin. Optimum chromatographic separation conditions with regard to sample load, binding and elution conditions have been determined using the AKTA Explorer-100 system (Amersham Pharmacia Biotech).

Molecular weight determination and N-terminal sequencing have been performed on the purified protein to confirm its identity. Polyclonal antibodies against the purified protein have been raised in a rabbit. The successive bleeds were tested for circulating antibodies by Western Blotting. Approximately 70 ml of antisera has been obtained, aliquoted and stored at −80° C. for future use.

The present invention comprises devices comprising oxalate-reducing enzyme activity, particularly devices used in urinary tract environments. Known devices can be modified by the methods taught herein, such as, but not limited to, by modification of the device surfaces and immobilization of oxalate-reducing enzymes on or within the devices, or attachment of coatings comprising oxalate-reducing enzyme activity on the devices, or polymeric matrix coatings wherein oxalate-reducing enzymes are immobilized or are released in a controlled manner into the surrounding environment. Hydrogel coatings are contemplated in the present invention. Hydrogel coatings can be used on the surface of devices or articles to improve biocompatibility, reduce bacterial adhesion, and reduce protein adsorption and encrustation. Hydrogel coatings, such as those formed from polyethylene glycol (PEG) cross-linking can be used as a matrix for drug delivery, as well as to immobilize biomolecules, such as enzymes The present invention comprises polydimethylsiloxane (PDMS) and polyurethane (PU) biomaterials, which are both relatively hydrophobic, hydrolytically stable, and exhibit good mechanical stability. Methods of the present invention comprise methods to immobilize oxalate-reducing enzymes in a PEG-albumin hydrogel matrix, and polymerizing the hydrogel matrix onto the surface of functionalized PDMS and PU elastomers.

Examples of elastomers commonly used as biomaterials in medical devices include Polydimethylsiloxane (PDMS), a silicone elastomer MDX4-4210, medical grade elastomer, (Dow Corning, Midland, Mich.) and polyurethane (PU), a thermoplastic polyurethane elastomer (Pellathane 2363-80A; Dow Chemical, Midland, Mich.). These elastomers undergo chemical surface modifications. Examples of chemical modifications are known in the art and described herein. An example of such modification follows. To covalently polymerize a hydrogel coating to the elastomer surface, a pre-treatment step may be performed to modify the PDMS and PU elastomer surface. This can be accomplished by applying a thin layer of gelatin derivatized with an aryl azide photoreactive chemical, which is covalently bound via irradiation with ultraviolet light, which links the PDMS surface C—H group to the azide group. The next step immobilizes one or more oxalate-reducing enzymes via polymerization of the hydrogel matrix in the presence of PEGylated-enzyme. An example of a “linker” comprises short-chained activated poly (ethylene glycol)-albumin chains that are first coupled to an enzyme, such as oxalate decarboxylase to form a-PEG/BSA/OXD, which then are polymerized into the PEG hydrogel matrix.

Methods are known in the art for derivatizing gelatin. For example, ATFB-gelatin is prepared by the addition of 8.3 mg (25 μmol) of 4-azido-2,3,5,6-tetrafluorobenzoic acid N-hydroxysuccinimide ester (ATFB-NHS) (Sigma Chemical Co., St. Louis, Mo.) in 0.5 ml methanol to 100 ml of 50 mM borate buffer (pH 8.6) containing 1% porcine gelatin A (Sigma Chemical Co.) Following overnight incubation at room temperature, the mixture is filtered and dialyzed against water for 24 hr. All procedures involving ATFB are performed in the dark. PDMS discs washed with methanol (30 min, ultrasonic) is immersed in 5 mg/ml ATFB-gelatin solution in methanol for 1 h at room temperature, removed and then dried for 2 hr at 40° C.

ATFB-gelatin-PDMS discs are then exposed to 254 nm ultraviolet light at a distance of 2 cm for 3 minutes in order to covalently bind the ATFB-gelatin to the PDMS surface via bonding of the PDMS surface C—H group and the ATFB azide group. Discs are washed with 1% sodium dodecyl sulfate (SDS) solution for 30 min at 80° C. to remove adsorbed (non-covalently bound) gelatin.

An example of methods for modifying polyurethane surfaces includes the following. PU devices are layered with a thin coating of 4-azidobenzoic acid-modified gelatin (AB-gelatin). AB-gelatin will be synthesized by the reaction of 4-azidobenzoic acid N-hydroxysuccinimide ester (AB-NHS) (Sigma Chemical Co., St. Louis, Mo.) using the same procedure followed for ATFB-gelatin on PDMS. AB is used since it generates a reactive intermediate that readily forms bonds with the polyurethane surface groups. The AB-gelatin-PU discs are exposed to 254 nm ultraviolet light at a distance of 2 cm for 3 minutes and washed with 1% SDS, similar to the ATFB-gelatin-PDMS discs.

An example of another chemical modification method of the present invention includes photopolymerization of vinyl monomers (e.g. acrylamide or n-vinylpyrrolidone), which can then immobilize enzymes and other biomolecules. Immobilization may be combined with the polymerization step, although some inactivation/degradation may occur during photopolymerization Photopolymerization methods can be used with transparent elastomers which provide the capability of forming the hydrogel within the lumen of the stent. Alternately, gamma irradiation technique could be used to activate all exposed surfaces of the device.

The present invention comprises methods of physical surface modification of the devices. The pulsed laser-assisted surface functionalization (PLASF™) technique can be used to functionalize the surface of both PDMS and PU. In addition to direct covalent attachment of the enzyme to the PLASF-functionalized biomaterial through glutraldehyde as the coupling agent, the enzyme can be linked to a hydrogel coating applied by the PLASF technique, using amine-terminated PEG as the target in PLASF chamber.

The present invention comprises methods for covalent enzyme immobilization in a hydrogel matrix. The surface modified elastomers, such as PDMS and polyurethane, are coated with an activated poly(ethylene glycol)-albumin-oxalate decarboxylase (a-PEG/BSA/OXD) solution. Activated PEG (a-PEG), which is polyethylene glycol bis(4-nitrophenyl carbonate), is synthesized from PEG (10 kDa; Sigma Chemical Co., St. Louis, Mo.) and 4-nitrophenyl chloroformate (Sigma) following the method of Fortier and Laliberté (49). To make a-PEG/BSA/OXD solution, OXD enzyme is mixed with bovine serum albumin (BSA) in borate buffer, pH 9.4, containing activated-PEG in ultrapure water. Surface modified PDMS and PU surfaces are dip-coated with the a-PEG/BSA/OXD solution and allowed to polymerize for 2 h at room temperature. As an alternative coating method, the PDMS and polyurethane surfaces may be spin-coated with the hydrogel solution. Different molecular weight PEG compositions may be used, the differences being related to the crosslinking density and hydrogel porosity, depending on the needed enzyme activity. For example, PEG compositions such as (4.6 kDa, 10 kDa, 20 kDa) can be used, depending on the optimum enzymatic stability and functionality for the specific purpose of the device.

The devices of the present invention, made using the methods disclosed herein, can also undergo sterilization prior to use or testing procedures to verify the claimed characteristics.

The present invention comprises devices that have at least one surface having one or more oxalate-reducing enzymes associated therewith. Both encrustation (extraluminal crusts) and incrustation (intraluminal crusts) are normally observed in long-term indwelling ureteral stents. As used herein, the terms encrustation and incrustation are used interchangeably unless otherwise noted. The present invention comprises methods for coating one or both of the outer and inner surfaces for an encrustation-resistant and/or incrustation-resistant stent. It may be that the reduction in oxalate concentration from an external enzyme coating of the stent will be sufficient for limiting the supersaturation within the lumen as well. It is noted that a normal stent has a peri-stent/intraluminal flow ratio of about 60:40. It is reported that a peri-stent obstruction reduces the ureteral flow more than an intraluminal obstruction: 74%, 43% and 25% for peri-stent versus 83%, 66%, 57% for intraluminal using the SF, 6F and 7F stents respectively. Thus, stents with complete intraluminal obstruction will still function because of extraluminal flow. Therefore, application of the enzyme coating is beneficial even if it is achieved on the outer surface of the stent only.

Chemical surface modification can be adapted to the entire stent when the technology is based on dip coating. This technology will coat both interior and exterior surfaces of the stent. PLASF™ surface functionalization utilizes a substrate rotator for holding different types of samples. This enables the deposition of a uniform outer coating on three-dimensional substrates. However due to the geometry of the laser-plume, some PLASF devices can be fitted to an extrusion apparatus. In vitro functional assessment of an enzyme-coated stent in urinary environment can be performed using the model developed by Tunney et al (50).

Briefly, a reaction vessel consisting of a perspex tank with a lose fitting lid, the contents of which are constantly agitated with two Teflon coated stirrers is placed in an incubator at 37° C. and an atmosphere equilibrated to 5% CO₂. Perspex columns are attached to the inside walls of the tank to allow positioning of a plastic grid 80 mm above the base of the tank. An aperture (diameter 40 mm) is cut from the center of the grid to allow exchange of solutions. Stent sections to be tested (length 50 mm) are suspended from the grid into the solution using plastic-coated paper clips. Several materials to be investigated under similar urinary conditions can be hung concurrently in this tank. The inhibition of surface encrustation and bacterial adhesion by enzyme-coated stent sections, as compared to non-coated stent sections can be assessed using the apparatus.

Compositions of the present invention comprise particles comprising oxalate reducing enzyme activity. Such particles may be nanoparticles or microparticles and are made from natural or synthetic materials, such as the polymers described herein. For example, polymeric matrix materials described herein, comprising entrapped oxalate reducing enzymes and optionally other active agents, may be formed in particles of such sizes.

Methods of the present invention comprise methods of making the devices and articles described herein comprising oxalate-reducing enzyme activity. Methods of the present invention also comprise methods of using the devices and articles described herein comprising oxalate-reducing enzyme activity. Methods comprise providing a device or article comprising oxalate-reducing enzyme activity to a subject, human or animal, so that at least one body fluid or tissue of the subject is in contact with the device or article, and reducing oxalate present in the body fluid or tissue. Methods for reducing oxalate in a human or animal comprise providing one or more particles comprising oxalate-reducing enzyme activity to a subject, human or animal, wherein at least one body fluid or tissue is in contact with the one or more particles, and reducing oxalate present in the tissue or body fluid environment of the subject. Body fluids include secretions of the body such as nasal or gastric secretions, saliva, blood, serum, urine, chyme or digestive matter, tissue fluid, and other fluid or semi-solid materials made by humans or animals. For example, one or more particles comprising oxalate-reducing enzyme activity can be administered orally to a human or animal and the oxalate-reducing enzyme activity reduces the oxalate present in the gut environment of the human or animal. Particles of the present invention may be mixed in food or other dietary materials and provided to a human or animal so that the oxalate-reducing enzyme activity of the particles is effective in the gut environment. Particles may also be mixed with foodstuffs or other materials in which oxalate is found and the oxalate-reducing enzyme activity of the particles reduces the oxalate present in the foodstuff or other materials.

Devices and articles of the present invention comprise oxalate reducing activity which is useful for reducing oxalate in the environment around the device or article. The oxalate reducing activity can be provided by chemically or physically attaching oxalate reducing enzymes to the device surface or to a coating which is applied to the device surface. Alternatively, the oxalate reducing activity can be provided by entrapping oxalate reducing enzymes in a polymeric matrix. The polymeric matrix may form the device or article, or may be a particle, or may be a coating. The oxalate reducing enzymes may remain within the polymeric matrix and reduce the oxalate that enters the matrix, or the oxalate reducing enzymes may be released in a controlled manner into the environment to reduce oxalate. Both of these methods are effective for immobilizing oxalate reducing enzymes and providing devices or articles with oxalate reducing activity.

An aspect of the present invention comprises devices comprising a polymeric matrix comprising at least one entrapped oxalate reducing enzyme. The polymeric matrix may be a coating applied to a medical device, or may be in the form of a particle. The polymeric matrix comprises natural polymers, synthetic polymers, biopolymers or combinations of two or more polymers, wherein the biopolymer comprises proteins, polysaccharides, mucopolysaccharides, heparin, heparin sulfate, heparinoids, dermatan sulfate, pentosan polysulfate, chondroitin sulfate, cellulose, agarose, chitin, carrageenin, linoleic acid, and allantoin, cross-linked collagen, fibronectin, laminin, elastin, cross-linked elastin, collagen, gelatin, hyaluronic acid, chitosan alginate, dextran, methylcellulose, DNA, RNA, other nucleic acid polymers, polylysine, and natural rubber, or combinations of one or more polymers. Synthetic polymers may include but are not limited to polyethylene glycol, polyvinyl alcohol, polyHEMA, polyacrylamide, polyacrylic acid, or methylethylcellulose. The oxalate reducing enzyme comprises oxalate oxidase, oxalate decarboxylase, or oxalayl-CoA decarboxylase, or combinations of one or more of these enzymes. It is to be understood that by use of the phrase “one or more enzymes” it is many enzyme molecules, active fragment molecules, etc., from one or more classes of enzymes. The polymeric matrix may be a coating on a stent, a catheter, a dialysis membrane, or an implant. During use of the device, the oxalate reducing enzyme may remain entrapped within the polymeric matrix, or may be released over time into a surrounding environment. The polymeric matrix may further comprise at least one active agent, including but not limited to, an antibiotic, antimicrobial agent, antifungal agent, antibacterial agent, anti-viral agent, antiparasitic agent, anesthetic, growth factor, angiogenic factor, tissue healing agent, adjuvant, antibody, or antibody fragment, or combination or mixtures of one or more agents. The oxalate reducing enzymes are active from the time the device is employed in an environment and for at least a year, or for at least four weeks, or for at least a week, or for at least four to six hours, and the length of activity may be determined by the environmental conditions and its effects on the enzymatic activity.

The present invention also comprises methods for reducing oxalate in an environment, comprising, providing a device comprising at least one oxalate reducing enzyme entrapped within a polymeric matrix to an environment comprising oxalate. The environment may be a fluid comprising oxalate, and fluids include nasal or gastric secretions, saliva, blood, serum, urine, chime or digestive matter, tissue fluid, and other fluid or semi-solid materials made by humans or animals. The environment may also be a site in the body, such as a urethra, or may be a site such as a container, as in fermentation tanks. The methods may also comprise providing devices which are particles. Polymeric microparticles or nanoparticles are contemplated by the present invention. It is to be understood that a plurality of particles may be provided in known drug delivery devices such as packaged in a gelatin or other kinds of capsule. Alternatively, a plurality of particles may be provided directly to a human or animal. Providing the devices of the present invention can be used for treatment of diseases or conditions wherein an unwanted amount of oxalate is present in an environment. One method of treating unwanted amounts of oxalate in humans or animals is to provide delivery devices comprises microparticles or nanoparticles of the present invention. Such particles may be provided with meals or in foodstuffs for ease of delivery.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Silicone Elastomer

Silicone elastomer (MDX4-4210, medical grade elastomer, Dow Corning, Midland, Mich., supplied by Factor II, Inc., Lakeside, Ariz.) was prepared following the manufacturer's instructions. Briefly, the elastomer was cast into 2-mm-thick sheets by curing the resin between acrylic plates separated by a 2-mm spacer. The prepared sheets were allowed to cure for 48 h at room temperature. Discs of 10-mm diameter were cutfrom the cured sheets with the use of a cork boring tool (Boekel, Feasterville, Pa.). The discs were extracted for 48 h in HPLC grade hexanes (Fisher Scientific Co., Pittsburgh, Pa.) to remove unreacted species.

### Surface Modification

The radio frequency plasma discharge (RFPD) system consisted of a bell-jar-type reaction chamber, a sample stand, a vapor inlet port, a vacuum system with liquid nitrogen cold-trap, an RF power generator (RF Plasma Products, Inc., model HFS 401 S) operating at a fixed frequency of 13.56 MHz with a maximum output of 500 W, and a matching network to coordinate the impedance of the plasma discharge with the RF power generator. Silicone elastomer discs were placed in the chamber, which was subsequently brought to a vacuum of 40 mtorr andpurged five times with Ar gas for 15 s at 1000 standard cm³/min. The final Ar gas purge was ignited to form plasma to clean and activate the silicone discs for 15 min at 50 mtorr, 50 watts power. Ultra pure water vapor was slowly added to the chamber with the use of a needle valve to replace the Ar gas in the plasma (15 min at 50 mtorr, 50 watts power). Following plasma treatment, discs were rinsed in 100% ethanol for 15 min, then reacted with a 2% (v/v) solution of 3-aminopropyltriethoxysilane (AMEO; Sigma Chemical Co., St. Louis, Mo.) in 95% ethanol for 45 min, and then rinsed with 100% ethanol. The discs were left to cure 16-24 h in ambient conditions.

### Surface Characterization

The surface of the plasma treated discs was characterized with the use of underwater captive air contact angle goniometry and x-ray photoelectron spectroscopy (XPS). Contact angle measurements were made in ultra pure water with the use of a Rame′-Hart A-100 goniometer. Three air bubbles of approximately similar size were introduced from below onto the silicone surface with the use of a bent micro syringe. The contact angle of each was measured immediately as the angle made between the air bubble and the surface of the modified silicone disc. A total of 21 measurements were made from 7 discs from the same plasma treatment, from which the average contact angle was determined. The change in surface elemental chemistry of unmodified, plasma-treated, and plasma-treated-AMEO-coated silicone elastomer was determined with the use of XPS. A low resolution survey scan and a high-resolution elemental scan was performed for each sample with the use of a Kratos Analytical XSAM 800 with a DS800 data-acquisition system. The Kratos was equipped with a Mg K_{α} X-ray source operating at a pass energy of 1253.6 eV, with the use of 15 kV and 9-mA current. Spectra were obtained at a take-off angle of 90° relative to the sample surface. Elemental analysis, providing relative atomic concentrations, was performed from the relative peak areas of the carbon (C1s), oxygen (O1s), silicon (Si2p), and nitrogen (N1s) peaks. The carbon peak was used to calibrate peaks for high-resolution scans.

### Pulsed Laser Assisted Surface Functionalization (PLASF™)

As an alternative surface modification method, the silicone elastomer surface was coated with a reactive silicone surface, using PLASF™ by Nanotherapeutics, Inc. (Alachua, Fla.). PLASF™ is a coating technique in which a medical grade silicone elastomer target (PDMS), placed in a vacuum chamber, is ablated with an excimer laser set to a specific wavelength. The ablated material forms a plume of polymer fragments, which are deposited onto a PDMS substrate. FIG. 2 shows a diagram of a typical experimental setup. A 2.5 cm diameter PDMS (cast/cured as above) target was mounted onto an aluminum stub. The PDMS target and a 6 cm×6 cm PDMS substrate were mounted to motors in the chamber and rotated during each deposition run so as to ensure uniform ablation. An excimer laser operating at 248 nm wavelength, at energies ranging from 100 to 250 mJ, was used for all experiments. The target was ablated for 5 min. The modified PDMS sample was then reacted with a solution of 2% AMEO in 95% ethanol for 1 hr. and then rinsed with 100% ethanol for 15 min (as for RFPD).

### Enzyme Immobilization

The oxalate-reducing enzyme, oxalate oxidase (OXO), from barley seedlings (Sigma Chemical Co., St. Louis, Mo.), was used. The enzyme was covalently bound to the activated silicone elastomer discs with the use of 2.5% (v/v) glutaraldehyde (Sigma Chemical Co., St. Louis, Mo.) in 0.01-M phosphate-buffered saline (PBS), pH 7.4. Plasma treated discs were placed into separate wells of a 24-well tissue culture plate. The discs were washed twice (5 min each) with PBS on a rocker bed under slight agitation. The 2.5% glutaraldehyde solution was added to each disc in a well, and the plate was incubated for 1 h at room temperature, under slight agitation. The discs were subsequently washed three times with PBS (5 min each), followed by two washes (5 min each) with 45-mM sodium succinate buffer at pH 4.0. The discs were then transferred to a clean tissue culture plate. To each disc, 1 ml of a 100-μg/ml oxalate oxidase solution, prepared in 45-mM sodium succinate buffer, pH 4.0, was added. The same amount of enzyme was added to a well with no disc, which served as a control reaction for enzyme activity analysis. The tissue culture plate was incubated on a rocker bed at 4° C. for 48 h at 20 rpm. Following incubation, the enzyme solution was aspirated and the discs were washed with sodium succinate buffer (5 min). The discs were tested for estimation of bound protein and enzymatic activity.

### Immobilized Enzyme Characterization

The OXO activity assay was determined by the method of Requena and Bornemann (Requena, L. et al., 1999, Biochem J 343:185-190). The assay is based on the colorimetric determination of H₂O₂ produced during degradation of oxalate by OXO. Discs with immobilized enzyme and 10 μg of the free incubated enzyme, in individual wells of a clean 24-well tissue culture plate, were incubated with 1 ml of 40 mM potassium oxalate in sodium succinate buffer, pH 4.0 at 37° C. (30 min) at 100 rpm. The samples were removed and boiled to quench the enzymatic reaction, and allowed to cool to room temperature. A volume of 0.5 ml of the above reaction mixture was added to a 1-ml disposable cuvette containing 0.5 ml 10-mM ABTS (2-2′azinobis-3-ethylbenzthiazoline-6-sulphonic acid) and 10 μl 2400-U/ml horseradish peroxidase, both in 45-mM sodium succinate buffer, pH 4. The cuvettes were covered and incubated at room temperature for 15 min. The absorbance was determined at 650 nm, with the use of a Shimadzu UV160 spectrophotometer, against a blank of buffered potassium oxalate, ABTS and horseradish peroxidase. A molar extinction coefficient of 10,000 M⁻¹ cm⁻¹ was used to calculate the activity of oxalate oxidase. One unit of activity was defined as the amount of enzyme required to degrade 1 mmole of oxalate per minute.

The amount of enzyme covalently immobilized onto the silicone elastomer surface was determined with the use of the QuantiPro™ BCA (bicinchoninic acid) assay kit (Sigma Chemical Co., St. Louis, Mo.). Silicone elastomer discs coated with OXO were incubated with 1 ml QP BCA working reagent tissue culture plate wells at 37° C. (2 h) then allowed to cool to room temperature, and the absorbance was read at 562 nm. The amount of immobilized enzyme was extrapolated from a standard curve of BSA solutions.

TABLE 1
Specific activity for oxalate-reducing enzyme
bound to surface modified silicone elastomer

| | | Total Protein | Specific Activity** |
|---|---|---|---|
| Treatment | Protein | (μg)** | U/mg protein |
| RFPD-AMEO | OXO | 24 ± 2 | 0.038 ± 0.006 |
| PLASF-AMEO | OXO | 24 ± 4 | 0.045 ± 0.012 |
| Free enzyme* | OXO | 0.7 ± 0.2 | 1.59 ± 0.26 |
| PLASF-AMEO | OXD | 23 ± 3 | 0.784 ± 0.200 |
| Free enzyme* | OXD | 7 ± 2 | 9.10 ± 1.81 |

*0.1 mL of 100 μg/ml enzyme in solution incubated for 48 hr at 4° C. as a control
**(mean ± SD)n = 9

The amount of immobilized protein and its specific enzymatic activity is presented in Table 1. Both OXO and OXD could be immobilized on to the silicone discs. On an average, about 24 μg of enzyme protein could be immobilized on an aminated 10 mm diameter silicone elastomer disc via glutaraldehyde bioconjugation. This corresponds to about 0.31 μg protein/mm².

Although both oxalate oxidase and oxalate decarboxylase were immobilized on the elastomer surface, the immobilized oxalate decarboxylase retained higher enzymatic activity. Also, since PLASF-AMEO treatment showed surface functionality and better specific activity for immobilized oxalate oxidase than RFPD, only the PLASF™ treatment was performed to immobilize oxalate decarboxylase.

### Example 2

### Modified Robbins Device

The ability of OXO-coated silicone elastomer to prevent calcium oxalate encrustation, was evaluated in a simulated urinary environment with the use of a continuous-flow encrustation model, a modified Robbins device (MRD). The device consists of a 23-cm acrylic block, enclosing a 1.0×1.5-cm channel, with tubing connectors at each end. A 0.5-cm-thick acrylic sheet, with 10 predrilled 1-cm-diameter holes, is secured to the acrylic block with screws; to prevent leaking, a rubber gasket is placed between the two acrylic sections. The discs are placed in plugs, each with a 2-mm-deep well, which are inserted into the 1-cm holes, allowing the inserted discs to remain flush with the acrylic top section, maintaining laminar flow within the device. Artificial urine (AU), the components of which are presented in Table 2, was pumped through the modified Robbins device at a rate of 0.8 ml/min. Two solutions of AU were prepared, one containing sodium oxalate and the other containing calcium chloride, to prevent calcium oxalate crystallization. The two solutions were mixed as the artificial urine was pumped to the MRD, resulting in a mixed solution with a pH of 6.0 and a final relative supersaturation of 8 as calculated by EQUIL (Wemess, P. G. et al., 1985, J. Urol 134:1242-1244).

Four OXO-coated discs and four control (no enzyme) discs were fitted into the sample plugs and incubated with AU for 6 days. One enzyme-coated and one control disc were removed at Days 2, 4, and 6 and rinsed with 45-mM sodium succinate buffer, pH 4.0. These discs were tested for enzymatic activity as previously mentioned and then tested for degree of encrustation. At Day 6 the remaining discs were rinsed with distilled water, allowed to dry, and prepared for scanning-electron microscopy (SEM) and energy-dispersive X-ray spectroscopy (EDS).

TABLE 2
Artificial Urine Composition, at pH 6 and
Final Relative Supersaturation of 8

| Substance | Final Conc.* | Ion | Final Conc. |
|---|---|---|---|
| NaCl | 105.50 | Na | 153.86 |
| KCl | 63.70 | K | 63.7 |
| Na₂SO₄ | 16.95 | Cl | 177.2 |
| NaH₂PO₄•H₂O | 3.23 | SO₄ | 20.8 |
| Na₃C₆H₅O₇•2H₂O | 3.21 | PO₄ | 3.23 |
| NaN₃ | 1.00 | Ca | 3.5 |
| MgSO₄ | 3.85 | Ox | 0.3 |
| Na₂C₂O₄ | 0.30 | Citrate | 3.21 |
| CaCl₂ | 4.00 | Mg | 3.85 |

*mmol/l

### Encrustation Assessment

The discs were rinsed with deionized water and treated with 1 ml of 0.05 M HCl at 4° C. overnight (15-20 h) to dissolve the encrusted material. The HCl solutions with dissolved oxalate were tested for oxalate concentration using the Sigma Diagnostics® Kit®for Oxalate (Sigma Chemical Co., St. Louis, Mo.). The oxalate concentration was used to quantitate the degree of encrustation at the disc surface. Structural and elemental analyses of disc encrustation were also performed with the use of scanning-electron microscopy (SEM) and energy-dispersive X-ray spectroscopy (EDS). Silicone elastomer samples were mounted on a ⅝ths in. aluminum stub with a piece of conductive tape. The samples and stub were coated with gold/palladium with the use of standard conditions. Samples were analyzed with the use of a JEOL 6400 microscope operating at 15-kV accelerating voltage, an aperture setting of 2-3 and a condensor lens current range of 9-10 nA. Digital image acquisition was performed with the use of the accompanying Oxford microanalysis hardware with a Link ISIS™ software package. EDS was performed for compositional analysis of the surface encrustation using the same hardware and software associated with the JEOL 6400 SEM.

TABLE 3
Quantitative assessment of degree of encrustation
on silicone elastomer discs incubated in artificial
urine in vitro using the modified Robbins device

| | Degree of Oxalate Encrustation* | | |
|---|---|---|---|
| | mmol/l | | Percent change |
| Time (Days) | Uncoated | OXD-coated | (%) |
| 4 | 0.265 | 0.125 | 53 |
| 9 | 0.505 | 0.265 | 48 |
| 14 | 1.344 | 0.667 | 50 |
| 21 | 2.832 | 2.423 | 14 |
| 28 | 3.093 | 3.014 | 3 |

*measured as oxalate concentration in dissolved encrusted material

SEM and EDS analysis: Elastomer discs were mounted on a ⅝ inch aluminum stub and coated with gold/palladium using standard conditions for morphological sample identification. Samples were analyzed using a JEOL 6400 microscope operating at 15 kV accelerating voltage, an aperture setting of 2-3 and a condenser lens current range of 9-10 nA. Digital image acquisition was performed using the accompanying Oxford microanalysis hardware with a Link ISIS™ software package. EDS was performed for compositional analysis of the surface encrustation using the same hardware and software associated with the JEOL 6400 SEM. SEM of both the uncoated control and the coated discs showed encrustation deposits that predominantly resembled calcium oxalate monohydrate crystals morphologically. The enzyme-coated discs removed from the Robbins device after incubating in artificial urine showed significantly fewer encrustation deposits than uncoated discs at days 6, 14 and 21.

### Example 3

### Bacterial Challenge

The ability of the immobilized OXD enzyme on PDMS to resist adhesion of two common uropathogens in artificial urine (AU) was investigated using the modified Robbins device. For each experiment, five OXD-coated PDMS discs, and five control (uncoated) PDMS discs were placed in the sterilized modified Robbins device. E. coli (ATCC 11775) or E. faecalis (ATCC 49477) were grown overnight in brain-heart infusion (BHI) broth at 37° C. The overnight culture of each bacterium was diluted to 10⁷ CFUs/ml in 25 ml AU supplemented with 2% BHI which was flowed in the device plugged at both ends, and incubated at 37° C. for 1 h. The bacterial-AU solution was removed and the device connected to the two AU stock solutions. Each AU stock solution was supplemented with 2% brain-heart infusion (BHI) to provide nutrients for bacterial growth. The BHI supplemented artificial urine was pumped through the device at 0.8 ml/min. At time points 1.5 h, 15 h, 24 h, and 48 h, one enzyme-coated disc, and one control uncoated disc was removed from the MRD and rinsed briefly in sterile 0.01 M phosphate-buffered saline (PBS), pH 7.4, to remove non-adherent and loosely adhering bacteria. The discs were placed in 3 ml of sterile PBS and gently sonicated on ice for two minutes. The bacteria adhering to the coated and control PDMS discs were enumerated by dilution plating on BHI agar.

The bacterial counts/disc of E. coli and E. faecalis adhering to OXD coated, and control uncoated discs for up to 48 h is shown in Table 4. After only 1.5 h of incubation in the modified Robbins device, the OXD enzyme-coated disc showed a reduction of 95% in the adhesion of E. faecalis. Reduction in E. faecalis adhesion was maintained for up to 24 h. The 48 h time point for E. faecalis was not determined. The number of adherent E. coli was much lower than those for E. faecalis, most likely representing differences in the intrinsic adhesion characteristics for each bacterial strain. In the first 15 h, E. coli showed slightly better adhesion to the OXD-coated disc; however, at the 24 h and 48 h time points adhesion of E. coli was significantly lower on the enzyme-coated discs as compared to the uncoated disc.

TABLE 4
Bacterial adhesion to silicone elastomer at different incubation times from artificial
urine supplemented with 2% BHI broth in a modified Robbins device.

| | | Adhering bacteria (×10⁶/disc) | | | |
|---|---|---|---|---|---|
| Bacteria | Sample | 1.5 h | 15 h | 24 h | 48 h |
| E. coli | Coated | 0.09 ± 0.04 | 1.4 ± 0.4 | 0.5 ± 0.04 | 1.9 ± 0.2 |
| | Uncoated | 0.06 ± 0.04 | 0.2 ± 0.05 | 1.2 ± 0.04 | 2.8 ± 0.4 |
| E. | Coated | 0.14 ± 0.05 | 22.2 ± 2.4 | 3.4 ± 0.1 | n.d. |
| faecalis | Uncoated | 2.13 ± 0.1 | 78.0 ± 4.5 | 33.0 ± 6 | n.d. |

### Example 4

### In Vivo Study

A preliminary study to determine whether the enzyme-coated discs would resist encrustation in vivo was performed in the rabbit model at the University of Western Ontario. Oxalate reducing enzymes, oxalyl-CoA decarboxylase and formyl-CoA transferase were adsorbed onto 5 mm² silicone (PDMS) discs. The presence of enzymatic proteins on the discs was confirmed by Enhanced Chemiluminescence (ECL) and Atomic Force Microscopy (AFM). Enzyme-coated (n=8) and control discs (n=8) were implanted into the bladders of New Zealand White rabbits. Following an indwelling period of 28 days, discs were recovered and the degree of encrustation on the polymer surface was evaluated by dry weight measurement, calcium determination by atomic absorption spectroscopy (AAS), and scanning electron microscopy (SEM). The mean dry weights of the coated and control discs following explantation were 0.389±0.209 gm and 0.668±0.291 gm, respectively (p=0.061). The mean weight of calcium in the coated and control discs, as determined by AAS was 354±216 mg and 637±296 mg, respectively (p=0.064). This in vivo study showed that the enzyme-coated biomaterial reduced encrustation in the urinary tract.

### Example 5

### Adsorption of Enzyme to SR

The oxalate-reducing enzymes used according to the subject invention can be adsorbed to a SR surface by immersing the substrate into a solution containing protein for several hours, at 37° C., with gentle stirring. Although protein adsorption onto SR is likely, one cannot expect to get high concentrations of the protein by this method. Ruggieri et al (81), have used an ionic surfactant, tridodecy-methyl-ammonium chloride (TDMAC), to complex heparin to hydrophobic catheter surfaces (latex, PVC, and PTFE). Although this method was successful for reducing bacterial adherence to the catheter surface, the heparin was found to leach off after about a week. Because of these inherent difficulties with these simple methods for enzyme immobilization, other, more permanent, methods of immobilizing the enzyme can be used. Alternative methods include:

1) SR can be activated within a plasma chamber and treated with ultrapure ammonia gas. The activated SR can then be dipped into a solution containing a coupling agent, such as glutaraldehyde or tris(hydroxymethyl)phosphine (THP). It has been shown that THP is a highly effective coupling agent for amine functionality, and it is less prone to hydrolysis than glutaraldehyde (Oswald, P. R. et al., 1998, Enzyme and Microbial Technology, 23:14-19). Finally, the modified silicone can be dipped into a solution containing the enzyme to link it to the coupling agent covalently through its amine basic residues. (Amino acid analysis shows that the oxalate reducing enzymes contain numerous such amine residues). Amine-containing side chains, such as lysine, arginine, and histidine, are typically exposed on the surface of proteins and can usually be derivatized with ease.

2) The methodology described by DiTizio et al (DiTizio, V. et al., 1998, Biomaterials, 19:1977-1884) can also be used. This method consists of applying a PEG-gelatin-liposome mixture to a silicone surface that has been pretreated with a thin layer of 4-azido-2,3,5,6-tetrafluorobenzoic (AFB) acid-modified gelatin. The AFB-gelatin can be synthesized, as outlined in their publication. The AFB-gelatin is immobilized on the silicone surface with UV light irradiation, and then the hydrogel mixture is applied by immersion of the coated biomaterial in an alkaline solution and crosslinked via reaction of the NPC-PEG (polyoxyethylene bis p-nitrophenyl carbonate) with the amine groups of the gelatin. This method can be adopted by replacing the liposome component with an oxalate reducing enzyme. The enzymes have amine groups which bind covalently with the NPC-PEG, along with the gelatin amine groups.

3) Polyacrylic acid (PAA) can also be grafted onto SR using argon-plasma technique (Langefeld, S. et al., 1999, Int. J. Art. Organs, 22:235:241). The enzyme can be covalently linked to the PAA by activation of the ends of grafted PAA chains with N(3-dimethylaminopropyl)-N′-ethylcarbodiimide hydrochloride.

### Example 6

### Immobilization of Oxalate Oxidase on Silicone Elastomer

A silicone elastomer surface was modified with RF plasma under Ar gas and then water vapor. Ar plasma resulted in a significant increase in surface hydrophilicity and relative oxygen content, as determined by contact angle and XPS analysis. Water-vapor plasma resulted in a further increase in the surface hydrophilicity and oxygen content, as compared to the Ar plasma treatment. Application of an AMEO coating to plasma treated silicone elastomer resulted in a relative decrease in surface hydrophilicity. Increased nitrogen content of the AMEO coated surface, as measured by XPS, indicated surface amination.

The active oxalate oxidase enzyme was immobilized on the aminated surface via glutaraldehyde bioconjugation. The immobilized OXO retained much of its native activity.

The effectiveness of the immobilized oxalate oxidase on PDMS discs toward prevention of calcium oxalate encrustation in the urinary environment was measured with the use of a modified Robbins device. Coating of the silicone discs with OXO led to 54% and 56% reduction in the concentration of oxalate on the encrusted material compared to the uncoated discs after 4 and 6 days of incubation in circulating artificial urine, respectively. The encrustation inhibition on the enzyme-coated surfaces was further confirmed by morphological and elemental analysis of the discs surfaces by SEM and EDS.

### Surface Modification

The captive air contact angle measurements and surface elemental compositions, as determined by XPS, are presented in Table 5 for the different plasma treatments.

TABLE 5
Captive Air Contact Angle Measurements and XPS Elemental
Surface Analysis of Plasma-Treated Silicone Elastomer

| | Contact Angle | Atomic Concentration (%) | | | |
|---|---|---|---|---|---|
| Plasma Treatment | (°)* | O | C | Si | N |
| Control (none) | 86 ± 4 | 27.9 | 46.5 | 25.6 | n/d |
| Ar | 35 ± 7 | 35.7 | 37.2 | 27.0 | n/d |
| H₂O | 23 ± 5 | 37.9 | 36.5 | 25.5 | n/d |
| AMEO coated | 96 ± 9 | 32.0 | 39.0 | 26.3 | 2.8 |

n/d = not detectable (<0.5%)
*(mean ± S.D.) n = 21)

Both Ar and H₂O plasma treatment produced surfaces with increased oxygen content and a corresponding decreased carbon content, as compared to control silicone elastomer, correlating with increasing hydrophilicity, as indicated by the decreased contact angle. H₂O plasma treatment, followed by AMEO coating, resulted in a strongly hydrophobic surface (contact angle of 96°), with oxygen and carbon amounts comparable to that of the control PDMS sample. The AMEO-coated surface was the only treatment that resulted in surface amination. The silicone content remained essentially unchanged after plasma treatment.

### Enzyme Immobilization

The amount of immobilized protein and its enzymatic activity, determined by the QP BCA protein estimation method, is presented in Table 6.

TABLE 6
Enzymatic Activity of Oxalate Oxidase
Immobilized on Silicone Elastomer

| | Total | | |
|---|---|---|---|
| | Protein | Activity | Specific Activity |
| Enzyme Sample | (μg)* | (μmoles/min)* | (U/mg protein)* |
| Immobilized OXO | 20.8 ± 3.6 | 0.0004 ± 0.0001 | 0.019 ± 0.003 |
| Free OXO^{a} | 10.0 ± 0.0 | 0.0004 ± 0.0002 | 0.040 ± 0.016 |

^{a}Oxalate oxidase in solution incubated at 4° C. for 48 h as a control.
*mean ± S.D., (n = 6)

On the average, 20.8 μg of oxalate oxidase (OXO) could be immobilized on an aminated 10 mm diameter silicone disc via glutaraldehyde bioconjugation, which corresponds to about 0.26 μg/mm2. The immobilized protein was found to be enzymatically active and retained 47.5% of its native activity, as determined with the use of the free enzyme (Table 6).

### Encrustation Assessment

Table 7 shows the amount of resolubilized oxalate, representing the degree of encrustation, from the silicone elastomer discs incubated in the MRD for 6 days. After 2 days of incubation in the MRD, there was very little encrustation deposition on either control or OXO-coated discs. After only 4 days, the OXO-coated silicone elastomer discs significantly inhibited encrustation deposition, with a more pronounced effect at 6 days.

TABLE 7
Quantitative Assessment of Degree of Encrustation
on Silicone Elastomer Discs Incubated in Artificial
Urine in vitro Using the Modified Robbins Device

| | | | Percent |
|---|---|---|---|
| | | Degree of | change vs |
| Sample | Time (days) | Encrustation^{a} | control (%) |
| | 2 | n/d | — |
| Control | 4 | 0.068 | — |
| | 6 | 0.115 | — |
| | 2 | n/d | n/d |
| OXO | 4 | 0.033 | 52 |
| | 6 | 0.053 | 54 |

n/d = not detectable
^{a}Measured as oxalate concentration in encrusted material dissolved in 0.05-M HCl.

Scanning-electron microscopy (SEM) of the control disc PDMS surface showed some encrustation deposits, whereas SEM of the corresponding OXO-bound PDMS discs revealed fewer encrustation deposits. The encrustation deposits were predominantly shown to morphologically resemble calcium oxalate monohydrate crystals. Electron-dispersive spectroscopy (EDS) of both surfaces confirmed that the encrustation deposits contained calcium. The compositional mapping for calcium confirmed that there was more encrustation deposition on control PDMS as compared to OXO-bound PDMS samples.

### Example 7

One milligram of oxalate decarboxylase is dissolved in 1 ml of distilled water. Separately, 10 ml of polyvinylpyrrolidone (20%) with N-vinylpyrrolidone (8%) is prepared in distilled water. Dropwise add oxalate decarboxylase solution into the PVP/NVP solution/colloid while stirring. Dip-coat a 5-Fr ureteral stent into the final solution. Vacuum dry the stent at 37° C. for 10 minutes and subject it to 0.1 Mrad of gamma irradiation. Place it in a desiccator to complete the drying process.

### Example 8

Three grams of HydromedD polymer were dissolved in ninety seven grams of 95% ethanol; then, an oxalate decarboxylase solution was mixed with the coating solution to achieve a final enzyme concentration of 0.1 mg/ml. A 1-cm stent segment was dip-coated with the coating material, and then air dried at 37° C. overnight.

### Example 9

A 1-cm, 6-Fr, polyurethane stent segment was dip-coated with 3% HydroslipC in 95% ethanol and cured at 80° C. for 10 min. A solution of 12% PEG (m.w.=10000) (w/v) and 0.22 M KCl was prepared and oxalate decarboxylase was added to achieve a final enzyme concentration of 0.1 mg/ml. Stent segment was immersed in the PEG/enzyme solution at 30° C. for 1 h. It was air dried at 37° C.

### Example 10

Five hundred micrograms of oxalate oxidase is dissolved in 1 ml of 10 mM phosphate buffer at pH 7 at 4° C. Dropwise add oxalate oxidase solution into cold 10 ml of a 3% polyethylene glycol polyurethane solution in a solvent system containing 5% distilled water and 95% ethanol while stirring. Dip-coat a 6-Fr ureteral stent into the polymer-enzyme solution. Dry the stent at 37° C. in a vacuum desiccator.

### Example 11

Two milligrams of the lyophilized oxalate decarboxylase are evenly suspended in 1 ml of cold 95% ethanol solution. Dropwise add oxalate decarboxylase solution into cold 9 ml of a polyethylene glycol polyurethane solution also in a 95% ethanol solvent while stirring. Dip-coat a 5-Fr ureteral stent in the enzyme-polymer suspension. Dry the stent in a vacuum desiccator at 50° C.

### Example 12

A 1-cm stent segment was dip-coated with 3% HydromedD in 95% ethanol and cured at 80° C. for 10 min. A solution of 12% PEG (m.w.=10000) (w/v) and 0.22 M KCl was prepared and 100 micrograms of oxalate decarboxylase was added. Stent segment was immersed in the PEG/enzyme solution at 30° C. for 1 h. It was air dried at 37° C.

### Example 13

A 5-French ureteral stent is dip-coated with a 3% polyethylene glycol polyurethane solution in a solvent system containing 5% distilled water and 95% ethanol. After drying at 80° C. for 10 minutes, it is dipped into 10 ml of 0.22 KCl solution containing 1 mg of oxalate oxidase and 1.2 grams of polyethylene glycol for 10 minutes. Dry the stent with absorbed enzyme in a vacuum desiccator at 30° C. for ten minutes. Immerse it in a 0.1% gluteraldehyde solution for 30 seconds and dry it at room temperature for 2 hours. Immerse it in large volume of 0.05% egg albumin solution at 4° C. for two hours. Wash off the free egg albumin in distilled water prior to drying it in a vacuum desiccator at 50° C.

### Example 14

A 6-French ureteral stent is dip-coated with a 2% HydromedD polymer solution in 95% ethanol while stirring. After drying at 80° C. for 30 minutes, it is dipped into 10 ml of 0.22 KCl solution containing 1 mg of oxalyl-CoA decarboxylase, 1 mg of formyl-CoA transferase and 1.2 grams of polyethylene glycol for 10 minutes. Dry the stent with absorbed enzyme in a vacuum desiccator at 30° C. for ten minutes. Immerse it in a 0.5% gluteraldehyde solution for 30 seconds and dry it at room temperature for 2 hours. Immerse it in large volume of 0.05% egg albumin solution at 4° C. for two hours with stirring. Wash off the free egg albumin in distilled water prior to drying it in a vacuum desiccator at room temperature. Then the stent is sterilized by ethylene oxide gas.

### Example 15

A 20-cm, 6-French polyurethane ureteral stent tubing was dip-coated with a mixture of 0.1% oxalate decarboxylase, 2.6% HydromedD polymer and 87% isopropanol while stirring. It was then dried at 80° C. for 20 minutes.

### Example 16

A 5-French ureteral stent is dip-coated in 10 ml of a polyvinyl alcohol (40%) solution containing 100 micrograms of oxalyl-CoA decarboxylase and 100 micrograms of formyl-CoA transferase. Dry the stent in a vacuum desiccator at 30° C. overnight. Immerse it in a 1% methylenebis(cyclohexyl-4,4′-isocyanate) solution for 20 seconds and dry it at room temperature under vacuum for 2 hours. Immerse it in large volume of 0.05% egg albumin solution at 4° C. for two hours with stirring. Wash off the free egg albumin in distilled water prior to drying it in a vacuum desiccator at room temperature.

### Example 17

A 6-French ureteral stent is immersed in a 10% methylenebis(cyclohexyl-4,4′-isocyanate) solution for two minutes. Drive off the solvent by placing it in a vacuum oven at 40° C. Dip it in an oxalate decarboxylase solution (0.01%) for two minutes. Air-dry it at room temperature. Dip-coat it with 3% HydroslipC and dry it at room temperature overnight.

### Example 18

A polysulfone low-flux dialyzer (Fresenius F6, Fresenius, and Bad Homburg, Germany) is coated by pumping 1 liter of mixture containing 0.1% oxalate decarboxylase, 0.5% HydromedS. 9.4% H₂O and 90% isopropyl alcohol through the dialyzer. Excessive liquid is immediately and quickly drained and the 70° C. purified dry air is pumped through the dialyzer in the reversed direction for 30 minutes to drive off isopropanol and water.

The coated dialyzer is aerated for 8 hours at room temperature and then packaged in an air permeable pouch. It is then ETO sterilized at 30° C. and 60% relative humidity for 60 minutes.

### Example 19

A 16-French latex Foley catheter is dip-coated with a mixture of 0.2% oxalate decarboxylase, 1.5% HydromedD polymer and 87% isopropanol while stirring. It was then dried at 80° C. for 20 minutes.

The coated catheter is aerated inside an air permeable pouch for two days at room temperature. It is then ethylene oxide sterilized at 30° C. and 60% relative humidity for 60 minutes.

### Example 20

The oxalate-reducing coated stents of the present invention are used in an animal model, the pig, for endourologic research This model is extensively used in development of commercially available urinary devices, and for study of encrustation deposition and biofilm formation onto ureteral stents.

In vivo studies in the pig model: Twelve 20-25 kg female farm pigs are used and both the experimental and control stents will be tested in the same animal. Enzyme-coated ureteral stents are cystoscopically positioned in one ureter as the experimental, and non-coated stents are placed in the other ureter as the control. Encrustation and microbial film formation on enzyme-coated and uncoated stents will be assessed after an indwelling period of 4 (n=6) and 12 (n=6) weeks. Assessment of the removed stents includes dry weight measurements, calcium determination by atomic absorption spectroscopy, chemical analysis by scanning electron microscopy and energy dispersive x-ray spectroscopy and microbial identification and quantification. Additionally, histological examinations of ureteral segments for determination of the degree of urothelium inflammation is performed.

Cystoscopy is performed and after identification of the ureteral orifices, one orifice will be cannulated with a guidewire. Under fluoroscopic guidance, an uncoated double J ureteral stent is placed (control group). Immediately following this, the second ureteral orifice is cannulated and a ureteral stent coated with oxalate-reducing enzymes is placed (experimental group). At the end of 4 weeks or 12 weeks, the animals are anesthetized for removal of the stents. Through a midline incision, both ureters are exposed and the stents removed and placed in sterile containers. A section of each ureter will be removed for histological examination. The animals then are immediately euthanized.

All ureters are assessed histologically and all ureteral stents undergo encrustation assessment for encrustation and microbial adhesion. The ureteric sections are examined under a light microscope to assess the degree of inflammation. Under sterile conditions, a one cm piece is cut from the proximal end of the retrieved stent for determination of microbial load. The stent section is sonicated in 5.0 ml of buffered saline to remove adherent bacteria. The dislodged bacteria is cultured, identified and quantified per ml of suspending buffer.

Encrustation assessment is performed on the dried stents. Specifically, dry weights of the stents is measured to determine the amount of encrustation, the amount of calcium in the encrustation is determined (by atomic absorption spectroscopy) and the chemical composition of the surface of the encrustation is determined by energy dispersive x-ray spectroscopy, based on scanning electron microscopy.

The stents coated with oxalate-reducing enzyme show less encrustation and incrustation than the uncoated stents, and the ureters showed less irritation with coated stents.

### Example 21

Microparticle compositions are made by adding 50 grams of silica particles, ranging from 5 to 10 microns are stirred into 100 ml of 1.5% sodium alginate and 0.5% oxalate oxidase solution. To make the oxalate oxidase solution, 0.5 g of oxalate oxidase is dissolved into 85 ml of distilled water. 1.5 g of sodium alginate is slowly stirred into the solution, and distilled water is added to bring the total volume to 100 ml. While shaking the mixture, spray the mixture into a large volume of 1% calcium chloride solution. Immediately collect the coated particles for by filtration Store the coated particles at a refrigerated temperature. Methods of using the particles include mixing the particles into a food source, such as cat food, and providing it to animals with hyperoxaluria.

### Example 22

Ten millilitres of fully swollen Sephadex G-20 particles of 35±15 microns are mixed with 20 ml of 1% alginate and 0.2% oxalate oxidase solution. While vibrating the mixture, pressure spray the mixture into 100 ml of 0.5% calcium chloride solution. Immediately collect the coated particles by gentle and brief centrifugation. Store the coated particles at refrigerated temperatures.

### Example 23

100 grams of fine silica particles of an average of 1 micron are stirred into 100 ml of a polyurethane solution containing 80% isopropyl alcohol, 16% water, 2% oxalate Decarboxylase and 1.5% polyurethane. While shaking and mixing the mixture, pressure spray the mixture into 60° C. dry air over a clean tray that is overlaid with a thin layer of fine sucrose powder. Immediately mix the powder with the particles and store at refrigerated temperature.

### Example 24

100 grams of porous silica gel particles of 250 to 500 microns are mixed with 100 ml of 0.5% oxalate oxidase. Lyophilize the slurry. Stir in the dry material into a 200 ml 1.8% sodium alginate and 0.8% oxalate solution quickly. While shaking and mixing the mixture, pressure spray the mixture into a large volume of 2% calcium chloride solution. Immediately collect the coated particles by filtration. Lyophilize the particles. Package the particles in size 2 gelatin capsules.

### Example 25

50 ml of fully swollen porous poly([allyl dextran]-co-N,N′-methylenebiscrylamide) particles of 50±p25 microns are packed into a MicroCheck™ filter funnel. Slowly pour 50 ml of 1% oxalate decarboxylase into the funnel and let excess fluid drain out by gravity. Briefly mix the particles into 100 ml of 2% alginate and 1.5% oxalate decarboxylase. While shaking and mixing the solution, pressure spray the solution into a large volume of 1% calcium chloride Immediately collect the coated particles by light vacuum filtration over the MicroCheck™ filter funnel. Lyophilize the particles. Package size 5 gelatin capsules with the particles. A method of use comprises treating animals with hyperoxaluria by adding one or more gelatin capsules to the food of the animal or administration by other oral routes.

### Example 26

50 ml of sterile and fully swollen porous poly([allyl dextran]-co-N,N′-methylenebiscrylamide) particles of 50±25 microns, which have large pores that accommodate proteins up to 20 million Daltons, are aseptically packed into a sterile MicroCheck™ filter funnel. Aseptically pour 50 ml of sterile filtered 1% oxalate decarboxylase into the funnel and let excess fluid drain out by gravity. Aseptically mix the particles into a sterile 100 ml solution of 2% alginate and 1.5% oxalate decarboxylase. While shaking and mixing the solution, aseptically pressure spray the solution into a large volume of sterile 1% calcium chloride and let sit for 5 minutes. Aseptically decant the supernatant and mix the precipitate with bifunctional PEG-epoxy to further seal the large pores to prevent release of the enzyme. Thoroughly wash away all unreacted bifunctional PEG-epoxy and aseptically pack the particles in hemodialysis cartridges.

It should be understood that the examples and embodiments described herein are for illustrative purposes only and that various modifications or changes in light thereof will be suggested to persons skilled in the art and are to be included within the spirit and purview of this application.

### REFERENCES

1. Tomson, C. R., et al., 1989, Clin Nephrol 32:87-95
2. Marangella et al., 1992, Nephron. 60:74-80
3. Hoppe et al., 1999, Kidney International 56:268-274
4. Fuse et al. 1994, J. Urol. 151:1703-1706;
5. Tiezer et al. 1998, J. Urol. 160:876-881;
6. Santin et al. 1999, Biomaterials, 20:1245-1251
7. Keane et al. 1994, Br. J. Urol. 73:687-691;
8. Robert et al. 1997, Urol. Int. 58:100-104
9. El-faqih et al. 1991, J. Urol. 146:1487-1491
10. Ramsay J W A 1987, Br. J. Urol. 1987, 66:66-70;
11. Denstedt et al. 1998, J. Endourol. 12:493-500
12. Tunney et al. 1996, Biomaterials, 17:1541-1547
13. Wollin et al. 1998, J. Endourol. 12: 101-111;
14. Teizer et al. 1998, Biomater. 43:321-330;
15. Reid et al. 1992, J. Urol. 148:1592-1594
16. Kulik E. and Y. Ikada, 1996, J. Biomed. Mater. Res. 30:295-304
17. D'Urso E M et al. 1995, Immobil. Biotechnol 23:587-595
18. Urban, M. W., M. T. Stewart, 1990, J Appl Polym Sci. 39:265-283;
19. Howard I. H. 1991, “Surface modification of polymers by radio frequency plasma, Ph.D. dissertation, University of Florida, page 76.
20. Lee, S. D., et al., 1996, J. Polym Sci Pol Chem 34:141-148
21. Mutlu, M. et al., 1991, J. Mater Chem 1:447-450;
22. Gaboury, S. R., et al., 1993, Adv Chem Ser 777-790; and
23. Rau, K. R., 2000, “Surface modification of biomaterials by pulsed laser ablation deposition and plasma/gamma polymerization” Ph.D. dissertation, University of Florida, Gainesville, Fla.
24. Lee, S. D., et al., 1994, J. Appl Polym Sci 54:1279-1287;
25. Lee, S. D. et al., 1996, Biomaterials 17:1599-1608;
26. Ksiue, G. H. et al., 1998, J Biomed Mater Res 42:134-147.
27. Langefeld, S. et al., 1999, Int. J. Artif Organs 22:235-241
28. Mason, M. et al., 2000, Biomaterials 21:31-36
29. Johnson J R et al. 1990, J. Infect. Dis. 162:1145-1150;
30. Gilchrist T. et al. 1991, Biomaterials 12:76-78
31. De Fife K M et al. 1999, J. Biomed. Material. Res. 44:298-307;
32. DiTizio, V et al. 1998, Biomaterials, 19:1877-1884.
33. Kotsira V P et al., 1997, Arch. Biochem. Biophys. 340:239-249.
34. Thompson C et al. 1995, Euphytica 85:169-172
35. Shimazono H., 1955, J. Biochem. 42:321-340.
36. Mehta A and A. Datta, 1991, J. Biol. Chem. 266:23, 548-53
37. Qyayle P R et al. 1961, Biochemical J. 78:611-615
38. Allison, M. J. et al. 1985, Arch. Microbiol. 141:47
39. Sidhu, H. et al., 1999 J. Am. Soc. Neph. 10:S334-S340.
40. Kleinschmidt, K. et al., 1993, In: Urolithiasis 2, Plenum Press, NY
41. Allison, M. J. et al. 1986, J. Nutr. 116:455-460;
42. Sidhu, H. et al. 1998, Lancet 352:1026-1029
43. Baetz A L and MJ Allison, 1989, J. Bact. 171:2605-2608;
44. Baetz A L and MJ Allison, 1990, J. Bact. 172:3537-3540;
45. Ruan Z S et al., 1992, J. Biol. Chem. 267:10537-19543
46. Abe, K. et al., 1996, J. Biol. Chem. 271: 6789-6793;
47. Lung H. Y. et al., 1994, J. Bact. 179:3378-3381;
48. Sidhu, H. et al., 1997, J. Bact. 179:3378-3381
49. Fortier G, et al. 1993, Biotechnology and Applied Biochemistry 17:115-130.
50. Tunney, M M, et al. 1996; Biomaterials 17:1025-1029.

What is claimed is:

1. A device, comprising a polymeric matrix comprising at least one entrapped oxalate reducing enzyme.

2. The device of claim 1, wherein the polymeric matrix is a coating applied to a medical device.

3. The device of claim 1, wherein the polymeric matrix is in the form of a particle.

4. The device of claim 1, wherein the polymeric matrix comprises natural polymers, synthetic polymers, biopolymers or combinations of two or more polymers.

5. The device of claim 4, wherein the biopolymer comprises proteins, polysaccharides, mucopolysaccharides, heparin, heparin sulfate, heparinoids, dermatan sulfate, pentosan polysulfate, chondroitin sulfate, cellulose, agarose, chitin, carrageenin, linoleic acid, and allantoin, cross-linked collagen, fibronectin, laminin, elastin, cross-linked elastin, collagen, gelatin, hyaluronic acid, chitosan alginate, dextran, methylcellulose, DNA, RNA, other nucleic acid polymers, polylysine, and natural rubber.

6. The device of claim 4, wherein the synthetic polymer comprises polyethylene glycol, polyvinyl alcohol, polyHEMA, polyacrylamide, polyacrylic acid, or methylethylcellulose, or a combination of one or more synthetic polymers.

7. The device of claim 1, wherein the oxalate reducing enzyme comprises oxalate oxidase, oxalate decarboxylase, or oxalayl-CoA Decarboxylase, or combinations of one or more enzymes.

8. The device of claim 1, wherein the polymeric matrix is a coating on a stent, a catheter, a dialysis membrane, or an implant.

9. The device of claim 1, wherein the oxalate reducing enzyme remains entrapped within the polymeric matrix.

10. The device of claim 1, wherein the oxalate reducing enzyme is released over time into a surrounding environment.

11. The device of claim 1, wherein the polymeric matrix further comprises at least one active agent.

12. The device of claim 11, wherein the at least one active agent is an antibiotic, antimicrobial agent, antifungal agent, antibacterial agent, anti-viral agent, antiparasitic agent, anesthetic, growth factor, angiogenic factor, tissue healing agent, adjuvant, antibody, or antibody fragment, or combination or mixtures of one or more agents.

13. A method for reducing oxalate in an environment, comprising, providing a device comprising at least one oxalate reducing enzyme entrapped within a polymeric matrix to an environment comprising oxalate.

14. The method of claim 13, wherein the environment is a fluid comprising oxalate.

15. The method of claim 14, wherein the fluid is nasal or gastric secretions, saliva, blood, serum, urine, chime or digestive matter, tissue fluid, and other fluid or semi-solid materials made by humans or animals.

16. The method of claim 15, wherein the fluid is urine.

17. The method of claim 13, wherein the device is a particle.

18. The method of claim 17, wherein a plurality of particles are packaged in a gelatin capsule.

20. The method of claim 17, wherein a plurality of particles is provided to a human or animal in conjunction with a foodstuff.

21. The method of claim 13, wherein the polymeric matrix comprises natural polymers, synthetic polymers, biopolymers or combinations of two or more polymers.

22. The method of claim 21, wherein the biopolymer comprises proteins, polysaccharides, mucopolysaccharides, heparin, heparin sulfate, heparinoids, dermatan sulfate, pentosan polysulfate, chondroitin sulfate, cellulose, agarose, chitin, carrageenin, linoleic acid, and allantoin, cross-linked collagen, fibronectin, laminin, elastin, cross-linked elastin, collagen, gelatin, hyaluronic acid, chitosan alginate, dextran, methylcellulose, DNA, RNA, other nucleic acid polymers, polylysine, and natural rubber.

23. The method of claim 21, wherein the synthetic polymer comprises polyethylene glycol, polyvinyl alcohol, polyEMA, polyacrylamide, polyacrylic acid, or methylethylcellulose, or a combination of one or more synthetic polymers.

24. The method of claim 13, wherein the oxalate reducing enzyme comprises oxalate oxidase, oxalate decarboxylase, or oxalayl-CoA decarboxylase, or combinations of one or more enzymes.

25. The method of claim 13, wherein the polymeric matrix is a coating on a stent, a catheter, a dialysis membrane, or an implant device.

26. The method of claim 13, wherein the oxalate reducing enzyme remains entrapped within the polymeric matrix.

27. The method of claim 13, wherein the oxalate reducing enzyme is released slowly into a surrounding environment.

28. The method of claim 13, wherein the polymeric matrix further comprises at least one active agent.

29. The method of claim 28, wherein the at least one active agent is an antibiotic, antimicrobial agent, antifungal agent, antibacterial agent, anti-viral agent, antiparasitic agent, anesthetic, growth factor, angiogenic factor, tissue healing agent, adjuvant, antibody, or antibody fragment, or combination or mixtures of one or more agents.
